(19) United States
(12) Patent Application Publication
(10) Pub. No.: US 2015/0253319 A1
(43) Pub. Date: Sep. 10, 2015

(54) Multi-unit plate for immunoblot analysis

(71) Applicant: Jiandi Zhang, Fairfax, VA (US)

(72) Inventor: Jiandi Zhang, Fairfax, VA (US)

(73) Assignee: YANTAI ZESTERN BIOTECHNIQUE CO. LTD., Yantai (CN)

(21) Appl. No.: 14/714,774

(22) Filed: May 18, 2015

Related U.S. Application Data
(63) Continuation-in-part of application No. 13/936,890, filed on Jul. 8, 2013, now Pat. No. 9,063,134.
(60) Provisional application No. 61/669,650, filed on Jul. 9, 2012.

(51) Int. Cl. G01N 33/543

(52) U.S. Cl. CPC G01N 33/54366

(57) ABSTRACT

This invention is provided for multi-unit plate with at least one unit comprised of a membrane, preferably nitrocellulose or PVDF membrane, on the surface of individual unit of multi-unit plate. These multi-unit plates are particularly well suited for high throughput immunoblot analysis including Zestern analysis.

### RELATED APPLICATIONS

This application is a continuation-in-part of U.S. patent application Ser. No. 13/936,890, filed on Jul. 8, 2013, which claims priority to U.S. Provisional Patent Application No. 61/669,650 filed on Jul. 9, 2012. Each of these applications is incorporated in its entirety into this application.

### FIELD OF INVENTION

The present invention relates to the field of immunoassay and more particularly, to devices and method for high throughput immunoblot analysis. Specifically, the present invention is of multi-unit plates and methods for performing Zestern analysis in a multi-unit format.

### BACKGROUND ART

Protein analysis is the foundation of modern biological research. Investigations of the expression and regulation of critical protein factors in biological processes and their applications in pharmaceutical and clinical studies provide vital information for experimental, pharmaceutical and clinical research of the pathogenesis of diseases and their prevention, diagnosis and treatments.

The recently patented Zestern technique (U.S. Pat. Nos. 8,293,487, 8,563,256 and 8,722,345) is an improvement of traditional methods of immunoblot-based protein analysis. While the protein samples are analyzed following a traditional immunoblotting process before detection, an additional step of elution is added in Zestern analysis to ensure the specificity of the assay. The antibody or antibody complex bound to the antigen of interest can be specifically competed out by competing molecule into elution solution. The amount of the eluted antibody or antibody complex in elution solution reflects reliably the amount of antigen of interest in the sample to be analyzed. The total amount of eluted antibody or antibody complex can be quantified directly in solution, representing another advantage of Zestern analysis over traditional immunoblotting methods.

While Zestern analysis demonstrates clear advantage over traditional immunoblot methods for its simplicity and suitability for high throughput analysis, it poses new demand for suitable devices, as existing devices for traditional immunoblot methods are not designed to meet the need of Zestern analysis, especially for the high throughput purpose.

In traditional immunoblot analysis, represented by Western blot analysis, several types of membranes have been used, and have been well optimized for immunoblot analysis. These membranes include both nitrocellulose membrane and PVDF membrane. Nonetheless, in traditional immunoblot analysis, the signal is detected on the very spot where the antibody or antibody complex bound to the antigen of interest on the membrane. This requires the membrane to be smooth and continuous to facilitate comparison of the detection results.

On the contrary, in Zestern analysis, antibody or antibody complex is liberated from the very spot where antibody or antibody complex bound to the antigen of interest by the competing molecule. The antibody or antibody complex is eluted individually from each spot for quantification. Clearly, in Zestern blot analysis, the membrane cannot be continuous among protein samples. It must be separated from each other to allow elution of antibody or antibody complex from individual protein sample, preventing cross-contamination of the signals from each other.

In Zestern analysis, for the membrane per se, there is no requirement regarding the shape or other physical characteristics of the membrane used, as detection of the signal from each sample is not being processed on the membrane.

Multi-well plate has been widely used in biochemical assays and immunoblotting assays including ELISA assay. These multi-well plates include 6, 24, 96, and even 1536 well plate. It can also be referred as microtiter plate, microplate, or microwell plate.

Multi-well plate for ELISA assay generally has protein binding capability at less than 1 μg/cm². In contrast, a typical membrane for traditional immunoblotting, regardless of nitrocellulose or PVDF membrane, has protein binding capability of 100 to 200 μg/cm². While ELISA plate has achieved success in ELISA assay, its low protein binding capacity limits its application in Zestern analysis.

Therefore, this invention provides solution to the unique demand of Zestern analysis for immunoblot analysis, especially for its application in multi-unit plate format.

### SUMMARY OF THE INVENTION

The present invention provides method and device with matching device for high throughput immunoblot analysis including Zestern analysis. The multi-unit plate of present invention includes at least one unit comprised of a membrane unit covering the surface of the unit of multi-unit plate.

In Zestern analysis, membrane used for individual sample is referred as individual membrane unit. An individual membrane unit is a piece of membrane, or pieces of membrane together associated with individual unit of the multi-unit plate, used for individual sample application. The individual membrane unit is separated from each other in the multi-unit plate. The individual membrane unit is eluted individually in the elution step for quantification of the individual sample.

There is no limitation of the shape, texture or even the continuation of the membrane for individual membrane unit. Multiple pieces of membrane can be considered as one individual membrane unit as long as they are within one unit of multi-unit plate.

The present invention for Zestern analysis is to use a multi-unit device in combination with a typical multi-well plate. The multi-unit plate is a plate with multi-units where at least in one unit of multi-unit plate comprised of one protrusion with an impermeable end, and the individual membrane unit is attached to the impermeable end of the protrusion of the plurality of the protrusions, preferably either PVDF or nitrocellulose membrane. The impermeable end of protrusion is not permeable to

Preferably, a plate of the present invention has a footprint of a standard multi-well plate. Preferably, the plurality of units of a plate of the present invention comprises 6n units arranged in a 2n by 3n array, where n is an integer greater than 0, the units preferably being arranged in rectangular packing. Preferred pluralities of units are the commonly known pluralities of units such as 6, 24, 96, 384 and 1536 units. More preferred are plates of 96 units and 384 units as these formats are most popular and have many available accessories including fluid handling accessories such as fluid-handling robots.

The multi-unit plate has at least one unit which is separated from other unit of the plurality of the units in multi-unit plate.

The present invention of multi-unit plate can fit into a typical multi-well plate with matching number of units and wells for Zestern analysis.

In one embodiment of the present invention, the individual unit in multi-unit plate can be individually addressable.

A multi-unit plate of the present invention is made of any suitable material. Suitable materials include but are not limited to ceramics, elastomers, epoxies, glasses, glass-ceramics, metals, plastics, polycarbonates, polydimethylsiloxane, polyurethane, polyethylenterephatalate glycol, polymers, polymethyl methacrylate, polystyrene, polyvinyl chloride, rubber, silicon, silicon oxide and silicon rubber.

In an embodiment of the present invention, the surface of the individual unit is made of any suitable material with protein binding capability comparable to nitrocellulose or PVDF membrane. The entire plate of the present invention can be made of one material, or it can be made of a number of different materials, for example, a plurality of layers or as a coated structure.

In an embodiment of the present invention, the surface of individual unit in the multi-unit plate is covered with individual membrane unit with smooth surface. In another embodiment of the present invention, the surface of individual membrane unit covering the surface of the individual protrusion of multi-unit plate may not be smooth.

In an embodiment of the present invention, there is no limitation of the shape or 3-dimisional structure of the individual membrane unit covering the surface of the individual unit of multi-unit plate as long as it allows accessibility of the samples of interest.

In an embodiment of the present invention, the individual membrane unit from multi-unit plate maybe treated before or after sample application to increase protein binding efficiency.

### BRIEF DESCRIPTION OF THE DRAWINGS

FIG. 1. Shows one embodiment of multi-unit plate. 1A, top view of the modified multi-unit plate 101. 1B, vertical side view of multi-unit plate 101; 1C, horizontal side view of the multi-unit plate; 1D, side view of individual unit of multi-unit plate including individual membrane unit covering a protrusion of multi-unit plate; 1E, the insertion of multi-unit plate into multi-well plate during sample application or elution steps. 101, multi-unit plate of present invention; 102, individual unit of multi-unit plate; 103, individual membrane unit covering the protrusion from multi-unit plate; 104, regular multi-well plate; 105, protein samples to be analyzed in the individual well of multi-well plate.

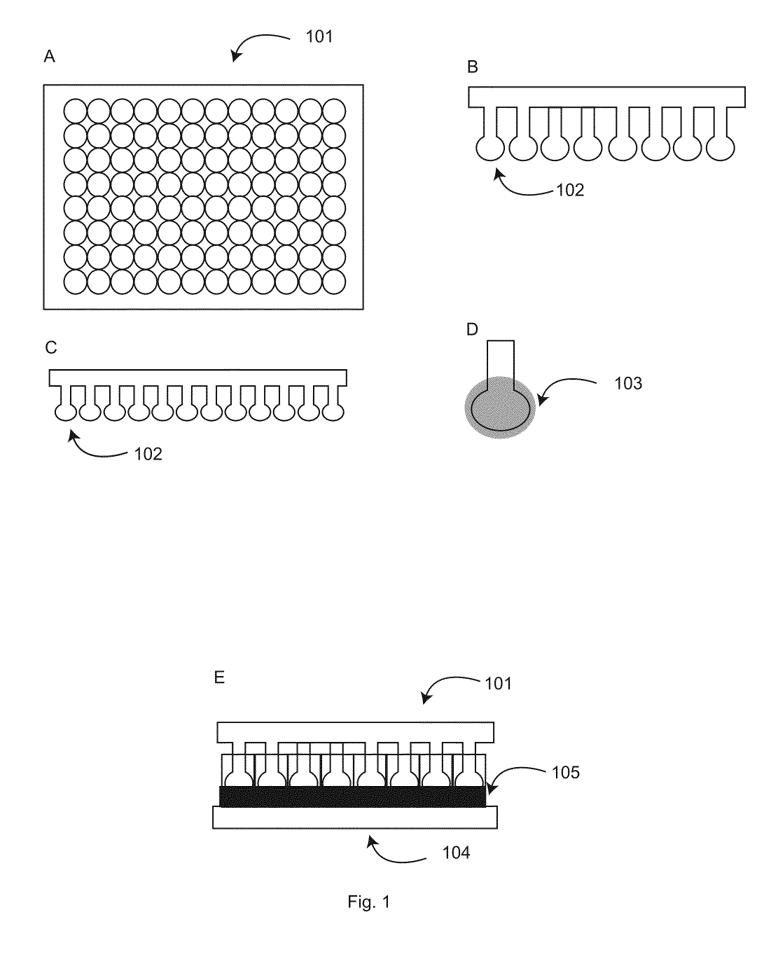

### DETAILED DESCRIPTION OF THE INVENTION

Unless defined otherwise, all technical and scientific terms used herein have the same meaning as is commonly understood by one of skills in the art to which this invention belongs.

The present invention provides device for immunoblot analysis including Zestern analysis. Zestern analysis distinguishes itself from traditional blot analysis including Western blot analysis by its simplicity and suitability for multi-unit format. The elution step in Zestern analysis also requires elution solution for individual sample to be physically separated from each other to avoid cross-contamination of the results. In other word, each sample must be applied to individual membrane unit, and elution solution for individual sample must be limited to individual membrane unit.

One embodiment of current invention 101 is shown in the drawing 1. A multi-unit-plate 101 is a plate with at least one unit 102 comprised of one protrusion with impermeable end covered by individual membrane unit 103, preferably either PVDF or nitrocellulose membrane.

The individual unit from multi-unit plate 102 is designed to increase membrane surface exposed to protein sample. At least in one unit of the multi-unit plate, the surface of protrusion is covered at least partly by individual membrane unit 103.

As used herein “membrane” is to be taken in its broadest context. A membrane can be any material within sufficient surface porosity to allow access by detection antibodies and a suitable surface affinity to bind antigen. All these materials may be used in suitable shapes, or they can be coated onto, or bonded or laminated, or simply attached to appropriate supporting materials, such as paper, glass, plastic materials. For example, membrane can be, but not limited to, nitrocellulose membrane or PVDF membrane.

The membrane associated with each unit is defined as individual membrane unit 103. An individual membrane unit can be a piece of membrane or pieces of membranes associated with individual unit of the multi-unit plate. There is no limitation of the shape, texture or even the continuation of the membrane from individual membrane unit.

Preferably, a plate of the present invention has a footprint of a standard multi-well plate. So, the multi-unit plate can fit inside a typical multi-well plate 104 as shown in the drawing. Preferably, the plurality of units of a plate of the present invention comprises 6n units arranged in a 2n by 3n array, where n is an integer greater than 0, the units preferably being arranged in rectangular packing. Preferred pluralities of units are the commonly known pluralities of units such as 6, 24, 96, 384 and 1536 units. More preferred are plates of 96 units and 384 units as these formats are most popular to find matching multi-well plate, and have many available accessories including fluid handling accessories such as fluid-handling robots.

The multi-unit plate 101 of the present invention is made of any suitable material. Suitable materials include but are not limited to ceramics, elastomers, epoxies, glasses, glass-ceramics, metals, plastics, polycarbonates, polydimethylsiloxane, polyurethane, polyethylenterephatalate glycol, polymers, polymethyl methacrylate, polystyrene, polyvinyl chloride, rubber, silicon, silicon oxide and silicon rubber.

In an embodiment of current invention, multi-unit plate 101 can be inserted into a typical multi-well plate 104 containing samples 105 in individual well of multi-well plate. Multi-unit plate 101 can be left in the air to dry, and individual unit 102 of multi-unit plate is inserted into appropriate containers to go through a typical immunoblot process including steps of blocking, primary antibody incubation, washing, secondary incubation and wash again.

The multi-unit plate 101 with immunocomplex bound on the individual membrane unit 103 of the protrusion of the plate is inserted into a typical multi-well plate 104 after a typical immunoblotting process. Elution solution containing appropriate competing molecule is used in each well of multi-well plate 104 to elute antibody or antibody complex from the individual membrane unit 103 on the surface of the protrusion of the multi-unit plate 101 for quantification of the signals.

In an embodiment of the present invention, the surface of the protrusion is covered by any suitable material with protein binding capability comparable to nitrocellulose or PVDF membrane. The entire plate of the present invention can be made of one material, or it can be made of a number of different materials, for example, a plurality of layers or as a coated structure.

Those skilled in the art will know how to prepare samples for immunoblot purpose. The samples include, but not limited to, a mixture of a chemical molecule, a peptide molecule, a protein molecule, an RNA molecule, a DNA molecule, a traditional antibody, e.g, two heavy chains and two light chains, a recombinant antibody or fragment, a bacteria cell, a virus particle, and a product comprising crosslinking any two or more of the above. The sample may be charged with appropriate sample buffer.

Those skilled in the art will know how to treat membrane for immunoblot. These practices include, but not limited to, direct application of samples to the membrane, or pre-wet the membrane portion of the multi-well plate with Ethanol, or Methanol, before sample application. The multi-unit plate with sample applied on the membrane is left in the air to dry before going through a typical immunoblot process.

The membrane can also be treated before or after sample application to increase protein binding efficiency. This practice includes, but not limited to UV crosslinking, or applying electric current on the membrane loaded with sample to increase protein binding efficiency.

Those skilled in the art will know how to process membrane with a sample applied on the surface for immunoblot process. These steps include blocking the membrane with blocking buffer, incubation with primary antibody, washing, incubation with secondary antibody and wash again to eliminate non-specific antibody binding to the membrane while preserving the immunocomplex bound on the surface of the membrane.

In one embodiment of present invention, after the sample application step but before the elution step, multi-unit plate 101 can be processed in a container other than a multi-well plate 104.

It is appreciated that many alternatives, modifications and variations will be apparent to those skilled in the art. Accordingly, the present invention is intended to embrace all such alternatives, modifications and variations that fall within the spirit and broad scope of the appended claims.

The following example of the method of invention is to further illustrate of the nature of the invention. It needs to be understood that the invention is not limited thereto.

### EXAMPLE 1

Samples of interest are prepared using 4×SDS buffer (Laemmli buffer). These samples are loaded into a regular 96 well plate at volume of 20 μl per well.

Multi-unit plate with protrusions covered with individual membrane unit arranging in the right order to ensure its insertion into 96 well plate, is inserted into 96 well plate containing sample of interest in individual well for 5 mins for sample application.

The multi-unit plate is removed from multi-well plate, and is left to dry for 20 mins before it is inserted into a large container containing 50 ml of blocking buffer and shake for 30 mins for blocking.

The multi-unit plate is incubated with primary antibody in blocking buffer at volume of 50 ml in a large container, and shakes overnight at 4° C. before it is washed three times with TBST buffer, each time for 5 mins at volume of 50 mls in a large container.

The multi-unit plate is incubated with secondary antibody in blocking buffer at volume of 50 ml in a large container, and shake overnight at 4° C. before it is washed again with TBST at 50 ml for three times, each time for 5 mins. The secondary antibody is labeled with Horseradish peroxidase as reporter enzyme.

While multi-unit plate being washed with TBST, a regular 96 well plate containing appropriate competing molecule in individual well of the plate is prepared at the volume of 150 μl/well.

After wash, the multi-unit plate is inserted into 96 well plate containing elution solution in individual well of the plate and shake for 10 mins.

The multi-unit plate is removed from 96 well plate, and the elution solution from individual well of 96 well plate is used for quantification in a typical chemiluminesence assay using a microplate reader.

What we claimed:

1. A multi-unit device for immunodetection assay comprising:
a multi-well plate with a plurality of wells; and
a complementary multi-unit plate sized to fit into the multi-well plate; the multi-unit plate comprising a plurality of unit of protrusion with impermeable end on the surface of the multi-unit plate, wherein the surface of the protrusion is covered by individual membrane unit.

2. The multi-unit plate of claim 1, where individual membrane unit is a membrane covering the protrusion of individual unit.

3. The multi-unit plate of claim 2, where individual membrane unit is one piece of membrane.

4. The multi-unit plate of claim 2, where individual membrane unit is more than one piece of membrane.

5. The multi-unit plate of claim 2, where the membrane is nitrocellulose or PVDF membrane.

6. The multi-unit plate of claim 1, wherein the membrane is treated before or after sample application to increase protein binding efficiency.

7. The multi-unit plate of claim 1, having a footprint of a standard multi-well plate to allow its insertion into a matching multi-well plate.

8. The multi-unit plate of claim 3, wherein said plurality of unit comprise 6n units arranged in a 2n×3n array, where n is an integer greater than 0.

9. The plate of claim 1, wherein said plurality of all units comprises individually addressable units.

10. The multi-unit plate of claim 1, wherein the membrane is sufficiently porous to allow penetration by the detection antibody.

11. The multi-unit plate of claim 6, wherein the membrane is coarse.

12. The multi-unit plate and multi-well plate of claim 1, comprising a material selected from the group consisting of ceramics, elastomers, epoxies, glasses, glass-ceramics, metals, plastics, polycarbonates, polydimethylsiloxane, polyethylenterephatalate glycol, polymers, polymethyl methacrylate, polystyrene, polyurethane, polyvinyl chloride, rubber, silicon, silicon oxide and silicon rubber.
